(19) United States
(12) Patent Application Publication
(10) Pub. No.: US 2019/0099120 A1
(43) Pub. Date: Apr. 4, 2019

(54) METHODS AND APPARATUS TO FACILITATE CONCUSSION SCREENING

(71) Applicant: Averia Health Solutions LLC, Oakton, VA (US)

(72) Inventors: Rohan Suri, Oakton, VA (US); Pranay Singh, Oak Hill, VA (US)

(21) Appl. No.: 15/722,818

(22) Filed: Oct. 2, 2017

(51) Int. Cl. A61B 5/00; A61B 3/11; A61B 3/14; A61B 3/00

(52) U.S. Cl. CPC A61B 5/4076; A61B 5/6898; A61B 3/112; A61B 3/14; H04B 1/3888; A61B 3/0025; A61B 5/6803; A61B 3/0008; A61B 3/0041

(57) ABSTRACT

Methods and apparatus to facilitate concussion screening are disclosed. Example apparatus disclosed herein include a housing, a mobile device bracket, a first mirror, a second mirror, and a light source. The housing has a viewing port. The mobile device bracket is disposed in the housing and configured to support a mobile device that has a display and a camera. The first mirror is disposed in the housing to reflect the display toward the viewing port. The second mirror is disposed in the housing to reflect the viewing port toward the camera. The light source is disposed in the housing.

### FIELD OF THE DISCLOSURE

This disclosure relates generally to medical devices, and, more particularly, to methods and apparatus to facilitate concussion screening.

### BACKGROUND

Athletes are often struck in the head during play. In some head injuries, the athlete's brain contacts the inner wall of the athlete's skull and is bruised, often referred to as a concussion. A severe concussion and/or repeated concussions may result in permanent physiological and/or mental damage (e.g., coordination problems, difficulty speaking, learning disability, etc.).

Concussions may be difficult to diagnose on the playing field or court and concussed athletes may not immediately feel symptoms of injury. Current accurate concussion diagnosis is performed by a healthcare professional in a hospital setting, often using specialized equipment. Thus, unevaluated asymptomatic yet concussed athletes may return to play, risking further brain injury. Conversely, a healthy athlete who has received a blow to the head but is not concussed may be unnecessarily removed from play in the interest of caution.

### SUMMARY

An example apparatus is disclosed. The example apparatus includes a housing, a mobile device bracket, a first mirror, a second mirror, and a light source. The housing has a viewing port. The mobile device bracket is disposed in the housing and configured to support a mobile device that has a display and a camera. The first mirror is disposed in the housing to reflect the display toward the viewing port. The second mirror is disposed in the housing to reflect the viewing port toward the camera. The light source is disposed in the housing.

Another example apparatus is disclosed. The example apparatus includes a stimulator, an image capturer, an elliptical arc extractor, and a velocity comparer. The stimulator provides a visual stimulus to a patient eye. The image capturer captures visual data of the patient eye in response to the visual stimulus. The elliptical arc extractor determines a first set of pupil locations at a first time and a second set of pupil locations at a second time based on the visual data. The velocity comparer determines a first pupil velocity and a second pupil velocity based on the first and second pupil location sets and the first and second times.

An example method is disclosed herein. The example method includes: providing, by executing an instruction with a processor, a visual stimulus to a patient eye; capturing, by executing an instruction with the processor, visual data of the patient eye in response to the visual stimulus; determining, by executing an instruction with the processor, a first set of pupil locations corresponding to a first time and a second set of pupil locations corresponding to a second time based on the visual data; and determining, by executing an instruction with the processor, a first pupil velocity and a second pupil velocity based on the first and second pupil location sets and the first and second times.

### BRIEF DESCRIPTION OF THE DRAWINGS

FIG. 1 is a front view of a first embodiment of a concussion testing device as disclosed herein.

FIG. 2 is a rear view of the concussion testing device of FIG. 1.

FIG. 3 is a first side view of the concussion testing device of FIG. 1.

FIG. 4 is a second side view of the concussion testing device of FIG. 1.

FIG. 5 is a top view of the concussion testing device of FIG. 1.

FIG. 6 is a schematic cross-sectional view of the concussion testing device of FIG. 1.

FIG. 7 illustrates a mobile device displaying a visual stimulus.

FIG. 8 illustrates the mobile device displaying response results to the visual stimulus of FIG. 7.

FIG. 9 is a block diagram of an example ocular movement tester implemented by the mobile device of FIG. 7.

FIG. 10 illustrates example visual data used by the ocular movement tester of FIG. 9.

FIG. 11 illustrates an example analysis process flow implemented by the ocular movement tester of FIG. 9.

FIG. 12 illustrates an example region detection process implemented by the ocular movement tester of FIG. 9.

FIG. 13 illustrates the example region detection process of FIG. 12 in operation.

FIG. 14 illustrates another example region detection process implemented by the ocular movement tester of FIG. 9.

FIG. 15 illustrates edge segments detected by the ocular movement tester of FIG. 9.

FIG. 16 illustrates a gradient vector corresponding to an example pixel analyzed by the ocular movement tester of FIG. 9.

FIG. 17 illustrates quantized gradient direction distributions of the edge segments of FIG. 15.

FIG. 18 illustrates an eye velocity determination process implemented by the ocular movement tester of FIG. 9.

FIG. 19 is a flowchart representative of machine readable instructions that may be executed to implement the ocular movement tester of FIG. 9.

FIG. 20 is a block diagram of an example computer capable of executing the instructions of FIG. 19 to implement the ocular movement tester of FIG. 9.

FIG. 21 is a rear view of a second embodiment of a concussion testing device.

FIG. 22 is a cross-sectional view of the concussion testing device of FIG. 21

FIG. 23 is a schematic cross-sectional view of the concussion testing device of FIG. 21.

The figures are not to scale. Wherever possible, the same reference numbers will be used throughout the drawings and accompanying written description to refer to the same or like parts. As used in this patent, stating that any part (e.g., a layer, film, area, plate, or assembly) is in any way positioned on (e.g., positioned on, located on, disposed on, or formed on, etc.) another part, means that the referenced part is either in contact with the other part, or that the referenced part is connected to the other part via one or more intermediate part(s) located therebetween. Stating that any part is in contact with another part means that there is no intermediate part between the two parts.

### DETAILED DESCRIPTION

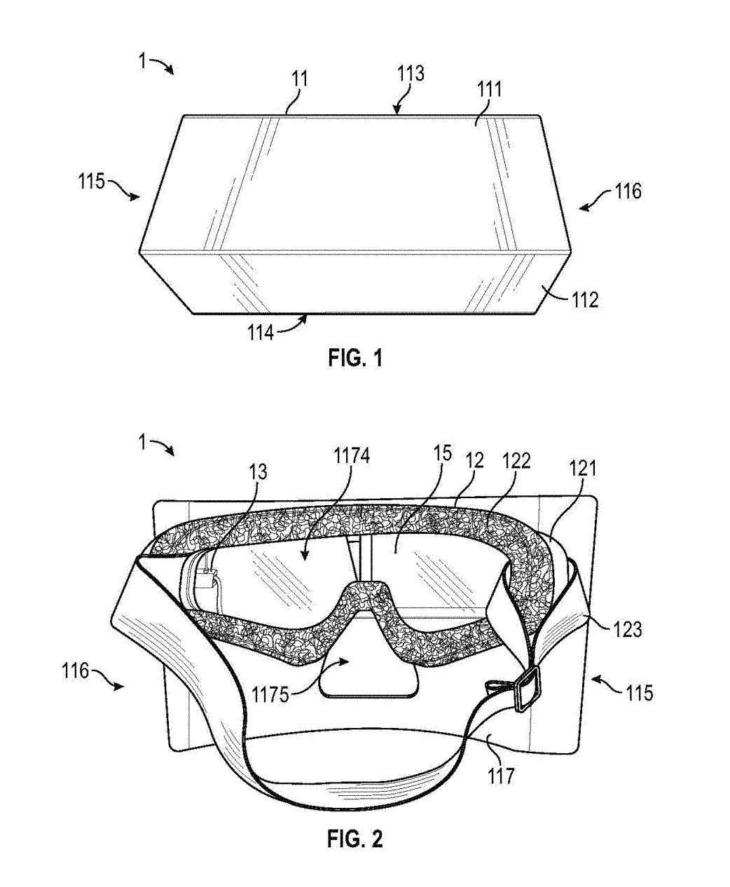

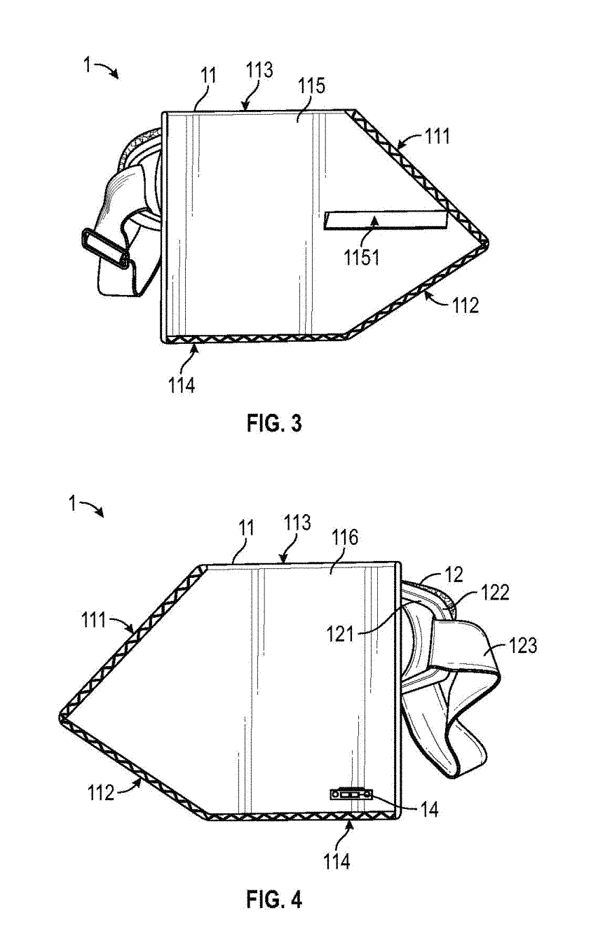

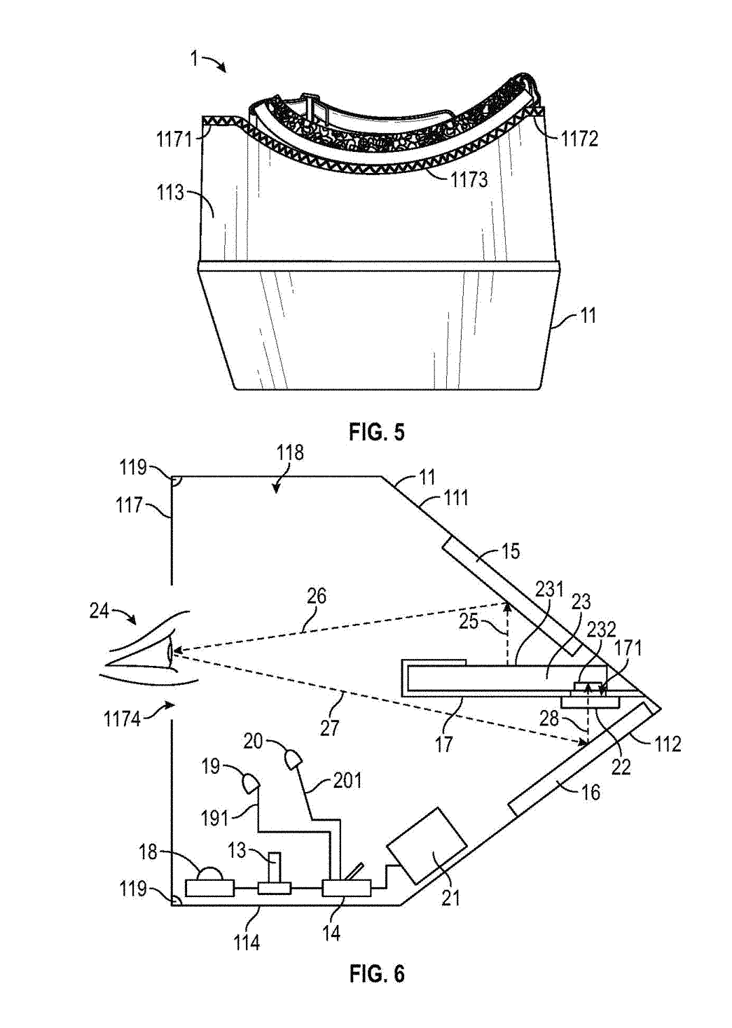

FIGS. 1-5 illustrate front, rear, first side, second side, and top views of a first example embodiment of a concussion testing device 1 as disclosed herein. In the illustrated examples of FIGS. 1-5, the concussion testing device 1 includes a housing 11, goggles 12, a potentiometer 13, a switch 14, and a first mirror 15. The housing 11 includes a first sloped wall 111, a second sloped wall 112, a top wall 113, a bottom wall 114, a first side wall 115, a second side wall 116, and a patient-facing wall 117. The patient-facing wall 117 includes a first straight portion 1171, a second straight portion 1172, and a curved portion 1173. The patient-facing wall 117 defines a viewing port 1174 and a nasal port 1175 in the curved portion 1173. The first side wall 115 defines a mobile device port 1151. The goggles 12 include a frame 121, a cushion 122, and a strap 123.

As shown in the illustrated examples of FIGS. 1-5, the top wall 113 is engaged with the first sloped wall 111, with the first side wall 115, with the second side wall 116, and with the patient-facing wall 117. The first sloped wall 111 is engaged with the second sloped wall 112, the first side wall 115, the second side wall 116, and with the bottom wall 114. The patient-facing wall 117 is engaged with the top wall 113, with the bottom wall 114, with the first side wall 115, and with the second side wall 116. In the illustrated examples of FIGS. 1-5, the housing 11 is formed of corrugated cardboard. It should be appreciated that the housing 11 may be formed of any sheet-type material (e.g., plastic, sheet metal, plywood, foam board, etc.). The walls 111, 112, 113, 114, 115, 116, 117 of the housing 11 may be engaged with one another with glue and/or with mechanical fasteners (e.g., staples, brads, nails, screws, pins, etc.). The walls 111, 112, 113, 114, 115, 116, 117 of the housing 11 may be integrally formed with one another from sheet material and folded to form the housing 11.

As shown in the illustrated examples of FIGS. 2-5, the frame 121 is engaged to the cushion 122. The strap 123 is engaged to the frame 121. The cushion 122 acts as a soft interface between a patient's face and the concussion testing device 1. The nasal port 1175 provides clearance for a patient's nose when using the concussion testing device 1. The cushion 122 further serves to substantially block light from entering the housing 11. In some examples, the cushion 122 is formed of elastomeric foam or the like. The strap 123 may be wrapped around a patient's head to hold the concussion testing device 1 firmly against the patient's face. In the examples of FIG. 205, the strap 123 is an elasticized band. It should be appreciated that the strap 123 may be formed of any lashing-type material (e.g., string, ribbon, leather, webbing, etc.).

In the illustrated example of FIG. 4, the switch 14 is externally carried by the second side wall 116. The potentiometer 13 is internally carried by the second side wall 116. It should be appreciated that the switch 14 and the potentiometer 13 may be carried by any of the walls 111, 112, 113, 114, 115, 116, 117 of the housing 11 internally or externally.

FIG. 6 is a schematic cross-sectional view of the concussion testing device 1 of FIG. 1. In the illustrated example of FIG. 6, the concussion testing device further includes a second mirror 16, a mobile device bracket 17, a diffused blue light-emitting diode (LED) 18, a first focused red LED 19, a second focused red LED 20, a battery 21, a red light filter 22. The housing 11 defines a cavity 118. The mobile device bracket 17 defines a camera opening 171.

In the illustrated example of FIG. 6, the first mirror 15 is engaged with and supported by the first sloped wall 111. The second mirror 16 is engaged with and supported by the second sloped wall 112. The mobile device bracket 17 is engaged with and supported by the first sloped wall 111. The mobile device bracket 17 is substantially perpendicular to the patient-facing wall 117. The diffused blue LED 18 is engaged with and supported by the bottom wall 114. Seams formed between the walls 111, 112, 113, 114, 115, 116, 117 of the housing 11 may be sealed with light-blocking sealant 119.

In the illustrated example of FIG. 6, the mobile device bracket 17 removably engages and supports a mobile device 23. The mobile device 23 includes a display 231 and a camera 232. The camera 232 is opposite the display 231 (e.g., rear-facing relative to the mobile device 23). The mobile device 23 is inserted into the mobile device bracket 17 via the mobile device port 1151. When inserted, the camera 232 is next to the camera opening 171. The mobile device bracket 17 is engaged with and supports the red light filter 22. The red light filter 22 covers the camera opening 171. In some examples, the red light filter 22 is formed of a translucent gel. Further, the mobile device 23 is removed from the mobile device bracket 17 via the mobile device port 1151.

In the illustrated example of FIG. 6, the battery 21 is in electrical communication with the switch 14. The switch 14 is in electrical communication with the first focused red LED 19 via a first supporting wire 191, with the second focused red LED 20 via a second supporting wire 201, and with the potentiometer 13. The potentiometer 13 is in electrical communication with the diffused blue LED 18. The first supporting wire 191 holds the first focused red LED 19 up in the cavity 118 near a first patient eye 24. The second supporting wire 201 holds the second focused red LED 20 up in the cavity 118 near a second patient eye (not shown).

In operation, the diffused blue LED 18 and the first and second focused red LEDs 19, 20 are energized by the battery 21 to provide a light source within the housing 11. The diffused blue LED 18 diffuses blue light throughout the cavity 118. The diffused blue LED 18 may be composed of multiple LEDs arranged in a strip. The potentiometer 13 may be used to adjust (e.g., regulate, control, etc.) the intensity of the blue light produced by the diffused blue LED 18. It should be understood that the potentiometer is optional. The first focused red LED 19 directs red light to the first patient eye 24. The second focused red LED 20 directs red light to the second patient eye. In other words, the first and second focused red LEDs 19, 20 illuminate the first patient eye 24 and the second patient eye.

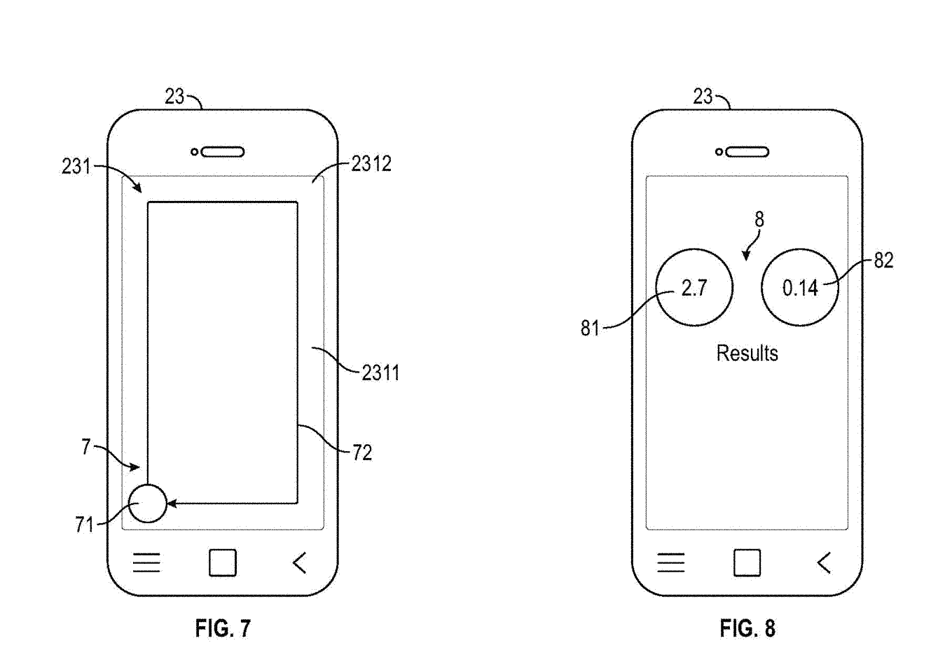

Further, in operation, a visual stimulus 7 is displayed on the display 231, as will be explained in greater detail below in conjunction with FIG. 7. The visual stimulus 7 bounces off the first mirror 15 to be observed by the first patient eye 24 and by the second patient eye via the viewing port 1174, as indicated by dashed lines 25, 26. In other words, the patient looks into the cavity 118 through the viewing port 1174 and sees the display 231 of the horizontally-mounted mobile device 23 via the first mirror 15. The camera 232 records responses of the first patient eye 24 and of the second patient eye to the visual stimulus 7 via the second mirror 16, as indicated by dashed lines 27, 28. In other words, the horizontally-mounted camera 232 observes how the patient's eyes react to the visual stimulus 7 via the second mirror 16. The use of red light is advantageous to create contrast between the respective irises and pupils of the first patient eye 24 and the second patient eye. The use of blue light is advantageous to constrict the pupils and to reduce strain on the first patient eye 24 and on the second patient eye. Additionally, the use of the red light filter 22 to filter light received by the camera 232 is advantageous to maintain the contrast between the irises and the pupils. It should be understood that it is advantageous to use the rear facing camera 23 of the mobile device 23 because the rear facing cameras of mobile devices often record at higher frame rates and resolutions than the front facing cameras of mobile devices.

It should be understood that the housing 11 can be of any shape that holds the first and second mirrors 15, 16, the LEDs 19, 20, and the mobile device 23 in positions relative to another as shown in FIGS. 1-6. In other words, the housing 11 may be in any configuration that holds the first and second mirrors 15, 16 diagonal to one another, supports the mobile bracket 17 approximately equally spaced between the first and second mirrors 15, 16, and supports the LEDs 19, 20 inside the housing 11.

FIG. 7 illustrates the mobile device 23 displaying the visual stimulus 7. In the illustrated example of FIG. 7, the visual stimulus 7 includes a stimulus shape 71 displayed on the display 231. It should be appreciated that the stimulus shape 71 may be any shape or image (e.g., ovular, circular, polygonal, zigzag, a tree, an animal, etc.). The use of images for the stimulus shape 71 may be advantageous where the concussion testing device 1 is used to evaluate children. In operation, the stimulus shape 71 moves in a pattern 72 about the display 231 to stimulate the first patient eye 24 and second patient eye of FIG. 6. In the illustrated example of FIG. 7, the pattern 72 is rectangular and the stimulus shape 71 moves near the edges 2311 and into the corners 2312 of the display 231. It should be appreciated that the pattern 72 may be any shape (e.g., ovular, circular, polygonal, zigzag, etc.).

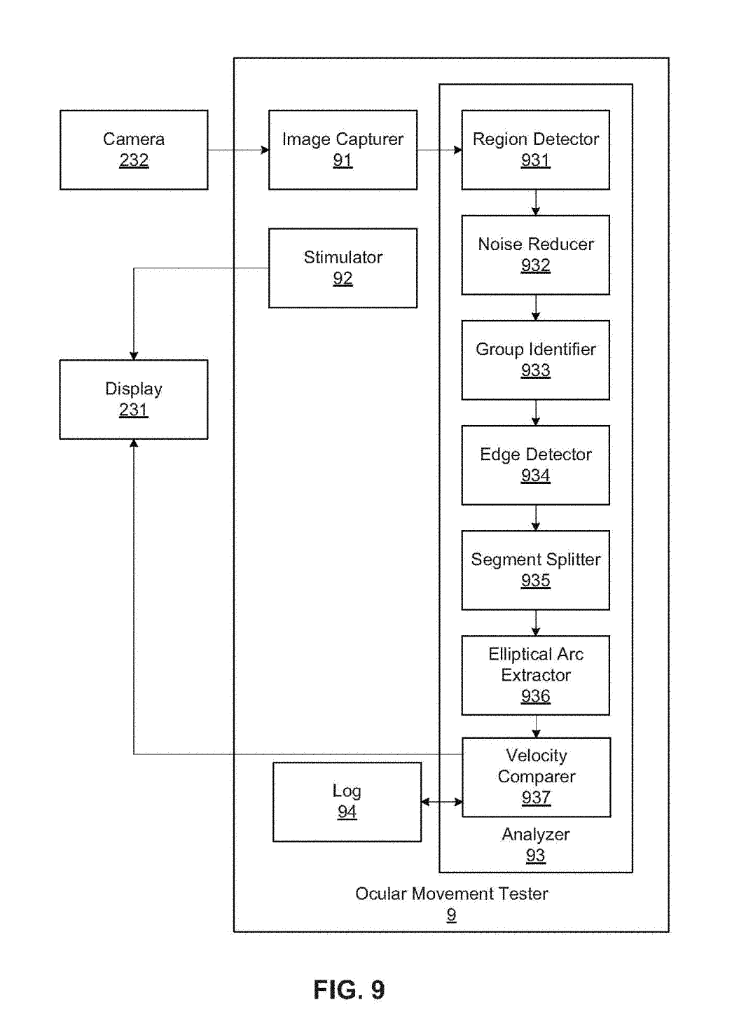

FIG. 8 illustrates the mobile device 23 displaying response results 8 to the visual stimulus 7 of FIG. 7. In the illustrated example of FIG. 8, the response results 8 include a first eye velocity 81 and a second eye velocity 82. In operation, the response results 8 are calculated by the mobile device 23, as will be explained in greater detail in conjunction with FIGS. 9-18, and are displayed on the display 231 of the mobile device 23.

FIG. 9 is a block diagram of an example ocular movement tester 9 implemented by the mobile device 23 of FIGS. 6-8. In the illustrated example of FIG. 9, the ocular movement tester 9 includes an image capturer 91, a stimulator 92, and an analyzer 93 and an eye velocity difference log 94. The analyzer 93 includes a region detector 931, a noise reducer 932, a group identifier 933, an edge detector 934, a segment splitter 935, an elliptical arc extractor 936, and a velocity comparer 937.

In the illustrated example of FIG. 9, the image capturer 91 is in communication with the camera 232 of the mobile device 23 of FIGS. 6-8 and with the region detector 931. The stimulator 92 is in communication with the display 231 of the mobile device 23 of FIGS. 6-8. The region detector 931 is in communication with the noise reducer 932. The noise reducer 932 is in communication with the group identifier 933. The group identifier 933 is in communication with the edge detector 934. The edge detector 934 is in communication with the segment splitter 935. The segment splitter 935 is in communication with the elliptical arc extractor 936. The elliptical arc extractor 936 is in communication with the velocity comparer 937. The velocity comparer 937 is in communication with the display 231 and with the eye velocity difference log 94.

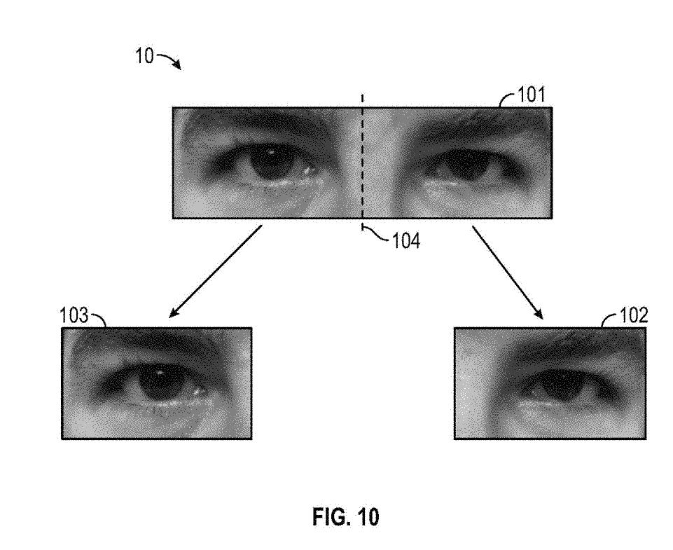

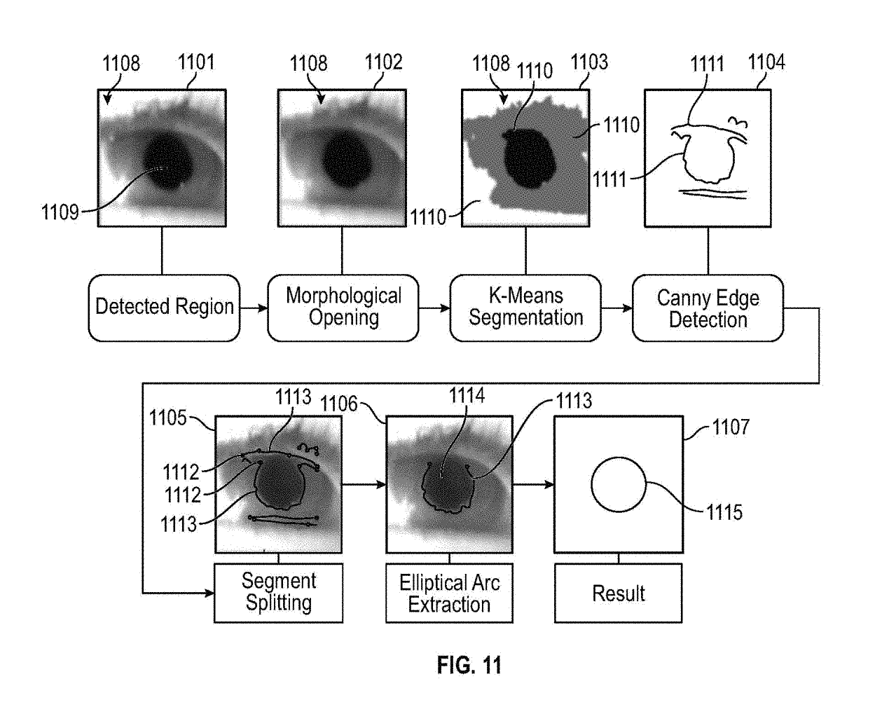

In operation, the stimulator 92 displays visual stimulus 7 on the display 231. Further in operation, while the visual stimulus 7 is shown on the display 231, the image capturer 91 captures visual data of the patient's eyes via the camera 232 in response to the visual stimulus 7, as will be explained in further detail in conjunction with FIGS. 10-11. Further, in operation, the analyzer 93 analyzes the visual data and generates the results 8 for display on the display 231, as will be explained in greater detail in conjunction with FIGS. 10-18. Additionally, in operation, the velocity comparer 937 may compare the results against the eye velocity difference log 94 to offer guidance via the display 231 on whether the patient is concussed.

FIG. 10 illustrates example visual data 10 used by the ocular movement tester 9 of FIG. 9. In the illustrated example of FIG. 10, the visual data 10 include a first frame 101, a first left eye image 102 (e.g., of the first patient eye 24), and a first right eye image 103 (e.g., of the second patient eye). In operation, the image capturer 91 of FIG. 9 captures the first frame 101 via the camera 232. Further, the image capturer 91 splits the first frame 101 into the first left eye image 102 and the first right eye image 103, as indicated by the dashed line 104. In other words, the image capturer 91 takes the first frame 101 with the camera 232 and generates the first left eye image 102 and the first right eye image 103 from the first frame 101. Further in operation, the image capturer 91 continues to capture additional frames at a regular frame rate (e.g. 24 frames per second, etc.) via the camera 232 and to generate additional left and right eye images respectively from the additional frames. Thus the image capturer 91 generates sequential left and right eye images separated by a known time period, the frame rate.

FIG. 11 illustrates an example analysis process flow 1100 implemented by the ocular movement tester 9 of FIG. 9. In the illustrated example of FIG. 11, the analysis process flow 1100 includes region detection 1101, noise reduction 1102, group identification 1103, edge detection 1104, segment splitting 1105, elliptical arc extraction 1106, and ellipsing 1107.

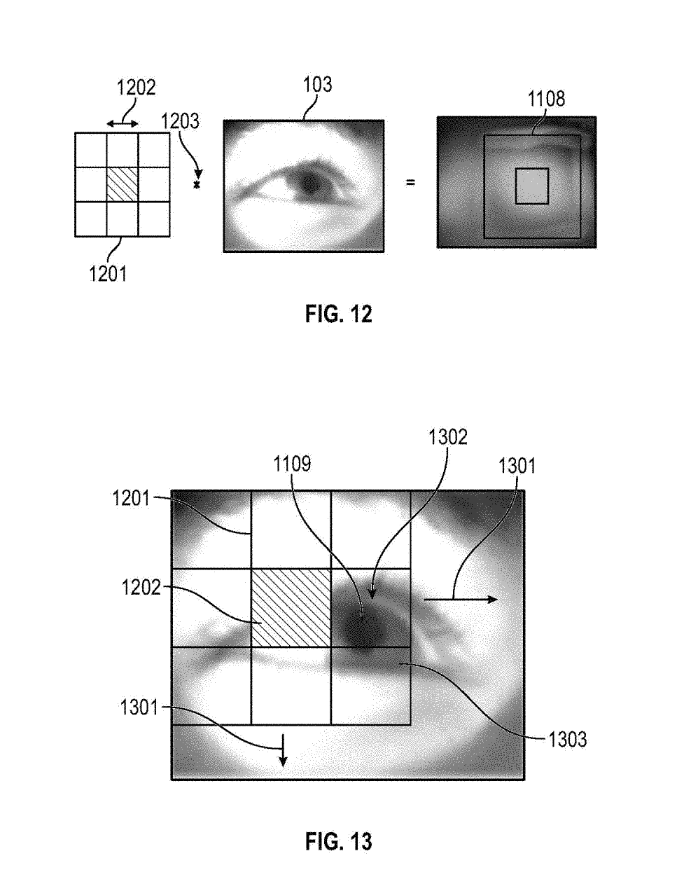

First, in operation during region detection 1101, the region detector 931 of the ocular movement tester 9 detects a region 1108 in each of the left and right eye images that contains a pupil 1109, as will be explained in greater detail in conjunction with FIGS. 12-14.

Second, in operation during noise reduction 1102, the noise reducer 932 of the ocular movement tester 9 reduces noise in the detected region 1108 (e.g., via a morphological opening as shown in FIG. 11).

Third, in operation during group identification 1103, the group identifier 933 of the ocular movement tester 9 identifies a plurality of groups 1110 within the detected region 1108 based on pixel colors of the detected region 1108 (e.g., via a K-means segmentation as shown in FIG. 11).

Fourth, in operation during edge detection 1104, the edge detector 934 of the ocular movement tester 9 detects edges 1111 of the groups 1110 (e.g., via Canny edge detection as shown in FIG. 11).

Fifth, in operation during segment splitting 1105, the segment splitter 935 of the ocular movement tester 9 finds corners 1112 along the edges 1111 (e.g., via curvature scale-space corner detection). Further, the segment splitter 935 splits the edges 1111 into a plurality of segments 1113 at the corners 1112. The corners 1112 and the plurality of segments 1113 are shown in greater detail in FIG. 15.

Sixth, in operation during elliptical arc extraction 1106, the elliptical arc extractor 936 of the ocular movement tester 9 determines and extracts an elliptical arc 1114 from the plurality of segments 1113, as will be explained in greater detail in conjunction with FIGS. 15-17.

Seventh, in operation during ellipsing 1107, the elliptical arc extractor 936 generates and fits an ellipse 1115 to the elliptical arc 1114. In the illustrated example of FIG. 11, the ellipse 1115 represents a pupil location of the pupil 1109 in the first right eye image 103. It should be understood that the elliptical arc extractor 936 generates and fits an ellipses to represent pupil locations of pupils in each left and right eye image.

FIG. 12 illustrates an example region detection process 1200 implemented by the ocular movement tester 9 of FIG. 9 during the region detection 1101 of FIG. 11. In the illustrated example of FIG. 12, the region detection process 1200 uses a Haar-like feature 1201 that has a predetermined radius boundary 1202. In the example of FIG. 12, the region detector 931 of FIG. 9 convolves the Haar-like feature 1201 over the first right eye image 103, as indicated by the convolution operator 1203 to yield the detected region 1108.

FIG. 13 illustrates the example region detection process 1200 of FIG. 12 in operation. In the illustrated example of FIG. 13, the region detector 931 of FIG. 9 moves the Haar-like feature 1201 across the first right eye image 103, as indicated by the arrows 1301 until the predetermined radius boundary 1202 covers (e.g., matches, pairs with) a dilatory region 1302. The dilatory region 1302 includes the pupil 1109 and an iris 1303 surrounding the pupil 1109. In operation, the region detector 931 also convolves the Haar-like feature 1201 over the first left eye image 102 to yield another detected region (not shown).

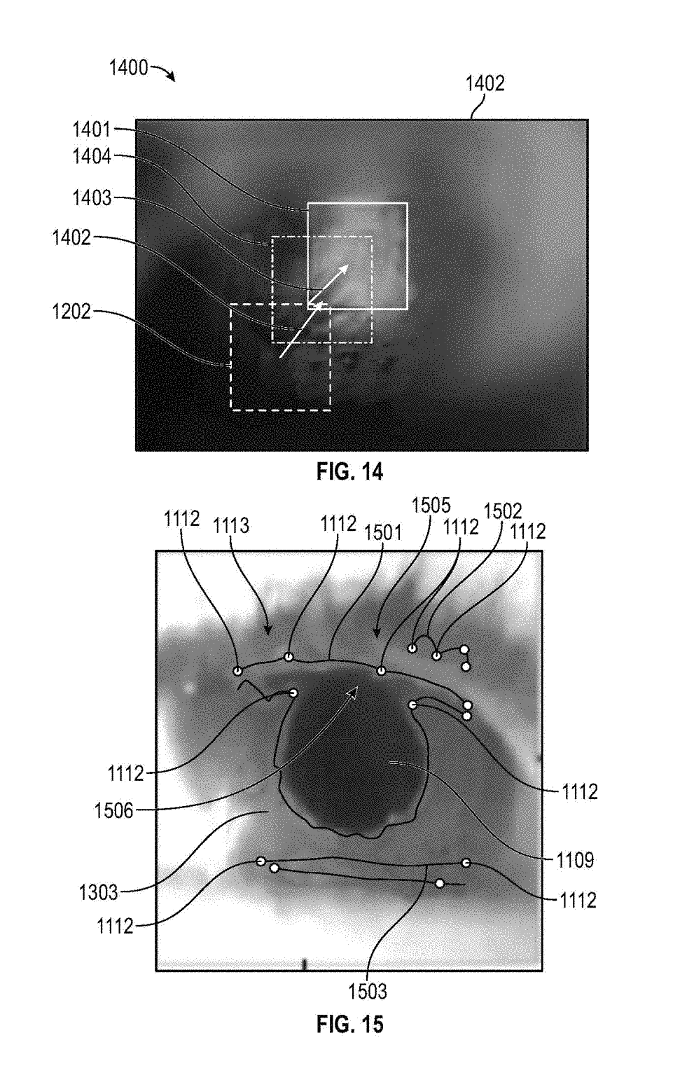

FIG. 14 illustrates another example region detection process 1400 implemented by the ocular movement tester 9 of FIG. 9. In the illustrated example of FIG. 14, the region detector 931 detects a second dilatory region 1401 in a second right eye image 1402 via a continuously adaptive mean (CAM) shift using the location of the predetermined radius boundary 1202 paired with a detected dilatory region of a previous right eye image. In the illustrated example of FIG. 14, arrows 1403 and a dot-dashed box 1404 indicate iterative calculations performed by the region detector 931 during the CAM shift process until convergence is found at the second dilatory region 1401.

FIG. 15 illustrates the plurality of segments 1113 detected by the ocular movement tester 9 of FIG. 9. In the illustrated example of FIG. 15, the plurality of segments 1113 particularly includes a first segment 1501, a second segment 1502, a third segment 1503, and a fourth segment 1504. As described above the segment splitter 935 locates corners 1112 along the edges 1111 and splits the edges 1111 at the corners 1112 to generate the plurality of segments 1113. In other words, each segment 1113 in the plurality of segments 1113 is separated from neighboring segments 1113 at the corners 1112.

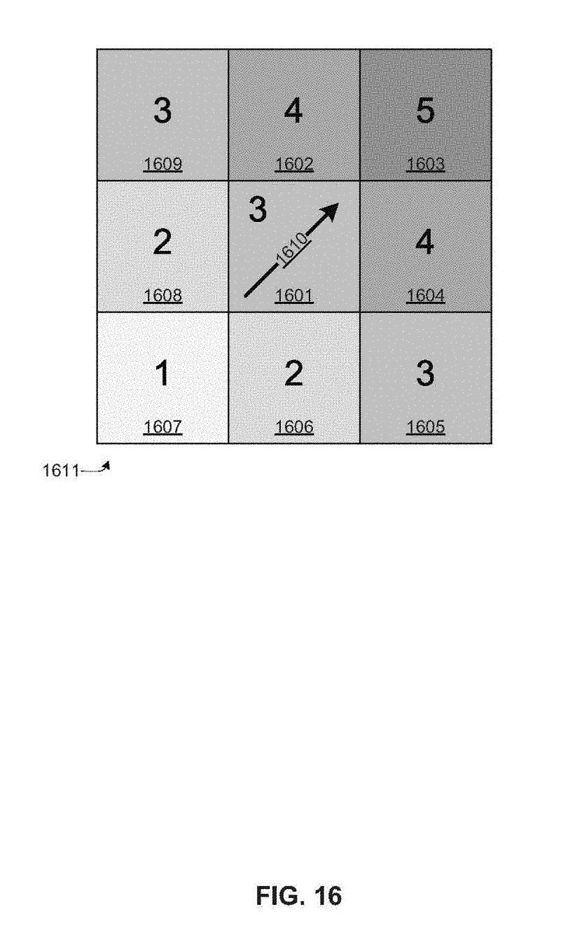

FIG. 16 illustrates a gradient vector 1610 corresponding to an example first pixel 1601 analyzed by the ocular movement tester 9 of FIG. 9. In the example of FIG. 16, the first pixel 1601 is neighbored and surrounded by neighboring pixels 1611. The neighboring pixels 1611 include a second pixel 1602, a third pixel 1603, a fourth pixel 1604, a fifth pixel 1605, sixth pixel 1606, a seventh pixel 1607, an eighth pixel 1608, and a ninth pixel 1609. It should be appreciated that each pixel along the segments 1113 has its own corresponding set of neighboring pixels.

In the illustrated example of FIG. 16, each of the pixels 1601, 1602, 1603, 1604, 1605, 1606, 1607, 1608, 1609 has a corresponding gray value. The seventh pixel 1607 has a gray value of 1. The eighth pixel 1608 and the sixth pixel 1606 each have a gray value of 2. The ninth pixel 1609, the first pixel 1601, and the fifth pixel 1605 each have a gray value of 3. The second pixel 1602 and the fourth pixel 1604 each have a gray value of 4. The third pixel 1603 has a gray value of 5. It should be understood that that any numbering scheme (e.g., binary, hexadecimal, etc.) may be used for gray value assignation besides or in addition to the integer gray values of the example of FIG. 16.

In the illustrated example of FIG. 16, the magnitude and the direction of the gradient vector 1610 are based on a first color difference between the respective gray values of the eighth pixel 1608 and of the fourth pixel 1604 and a second color difference between the respective gray values of the sixth pixel 1606 and of the second pixel 1602. Thus, in the example of FIG. 16, the first color difference yields a value of 2 and the second color difference also yields a value of 2. Therefore, in the example of FIG. 16, the gradient vector 1610 has a magnitude of approximately 2.828 at approximately a 45 degree direction. It should be understood that, in operation, the elliptical arc extractor 936 determines a gradient vector for each pixel along the segments 1113.

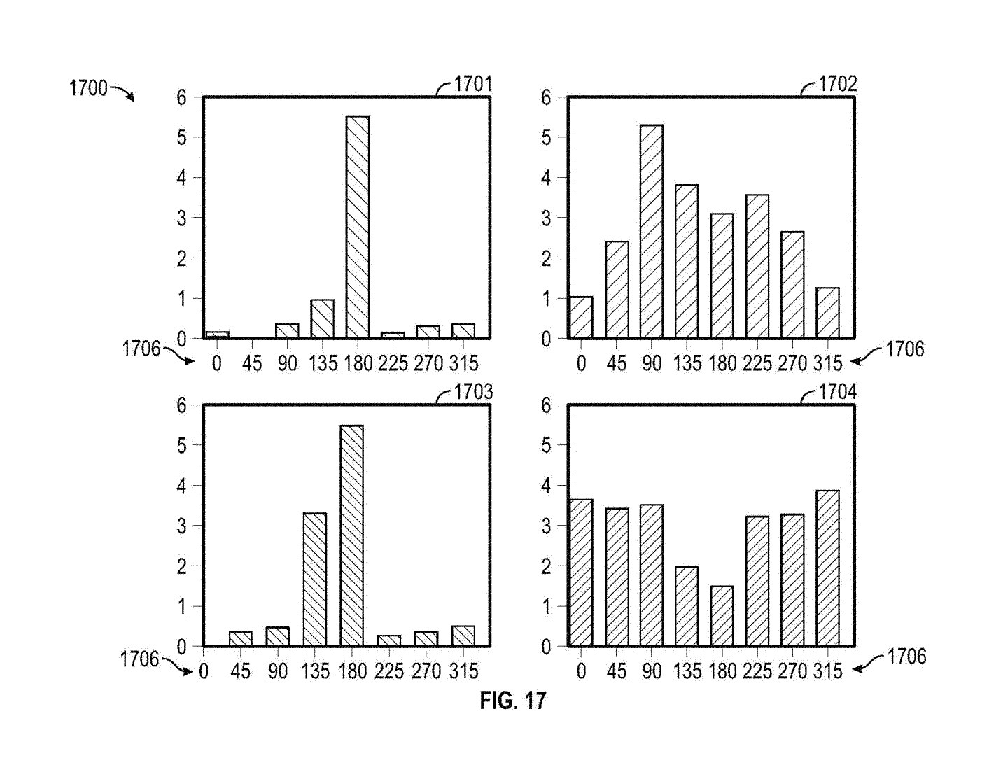

FIG. 17 illustrates quantized gradient direction distributions 1700 of the segments 1113 of FIG. 15. In the illustrated example of FIG. 17, the quantized gradient direction distributions 1700 include a first quantized gradient direction distribution 1701, a second quantized gradient direction distribution 1702, a third quantized gradient direction distribution 1703, and a fourth quantized gradient direction distribution 1704. The first, second, third, and fourth quantized gradient direction distributions 1701, 1702, 1703, 1704 respectively correspond to the first, second, third, and fourth segments 1501, 1502, 1503, 1504 or FIG. 15.

In operation, the elliptical arc extractor 936 sorts the gradient vectors of each pixel along the segments 1113 into the closest-matching of the degree value bins 1706 (e.g. 0, 45, 90, 135, 180, 225, 270, 315, etc.). For example, the elliptical arc extractor 936 would sort a gradient vector with a 215 degree direction into the 225 degree value bin 1706. In operation, the elliptical arc extractor 936 then compiles the magnitudes of the degree value-sorted gradient vectors of to generate a quantized gradient direction distribution. Rephrased, the elliptical arc extractor 936 aggregates the magnitudes of gradient vectors that have approximately like degree directions for each pixel along a segment 1113 to generate a quantized gradient direction distribution for the segment 1113. Although quantized gradient direction distributions are shown for only the first, second, third, and fourth segments 1501, 1502, 1503, 1504 in the illustrated examples of FIGS. 15 and 17, it should be appreciated that the elliptical arc extractor 936 generates respective quantized gradient direction distributions for each of the plurality of segments 1113.

In the illustrated examples of FIGS. 15 and 17, the first segment 1501 is relatively straight with a light region 1505 above and a dark region 1506 below. The light region 1505 and the dark region 1506 are approximately parallel bands. In other words, darker pixels of the dark region 1506 are located approximately “due south” (e.g., 180 degrees) of lighter pixels of the light region 1505 along the first segment 1501. Thus, the first quantized gradient direction distribution 1701 shows a high 180 degree distribution. It should be understood that the first segment 1501 is relatively straight because the dark region 1506 is relatively parallel to the light region 1505. Similarly, the third quantized gradient direction distribution 1703 shows a high 180 degree distribution because the third segment 1503 is also relatively straight. Further, the second quantized gradient direction distribution 1702 shows a curved distribution because the second segment 1502 is parabolic.

In the illustrated example of FIGS. 15 and 17, the fourth segment 1504 is nearly circular, with the light iris 1303 about the dark pupil 1109. In other words, lighter pixels of the iris 1303 surround darker pixels of the pupil 1109 in all directions. Thus, the fourth quantized gradient direction distribution 1704 shows relatively even distribution over all directions (e.g., 0 through 315). In other words, each pixel along the fourth segment 1504 has an approximately opposing counterpart pixel with correspondingly approximately opposite gradient vectors. It should be understood that the fourth segment 1504 is relatively circular because the lighter pixels of the iris 1303 encircle the darker pixels of the pupil 1109. Thus, a relatively even quantized gradient direction distribution indicates (e.g., is reflective of) a segment 1113 that is elliptical.

In operation, the elliptical arc extractor 936 filters out segments 1113 that yield uneven quantized gradient direction distributions. In the illustrated examples of FIGS. 11, 15, and 17, elliptical arc extractor 936 filters the plurality of segments 1113 to yield the fourth segment 1504 as the elliptical arc 1114.

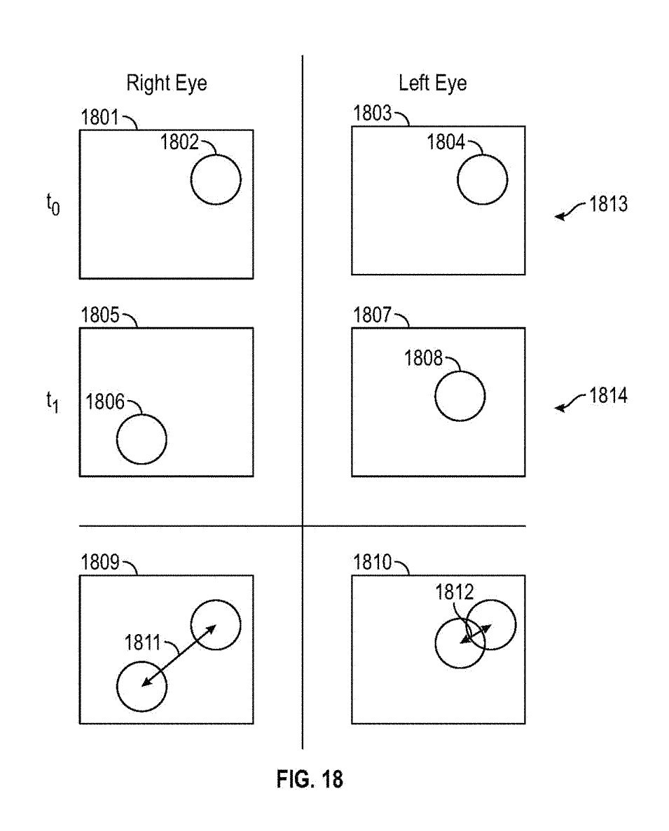

FIG. 18 illustrates an eye velocity determination process 1800 implemented by the ocular movement tester 9 of FIG. 9. In the illustrated example of FIG. 18, a first right eye image 1801 captured by the image capturer 91 at a first time t₀ includes a first right pupil ellipse 1802. A first left eye image 1803 captured by the image capturer 91 at the first time t₀ includes a first left pupil ellipse 1804. The first right and left pupil ellipses 1802, 1804 may be collectively referred to as a first pupil location set 1813. A second right eye image 1805 captured by the image capturer 91 at a second time t₁ includes a second right pupil ellipse 1806. A second left eye image 1807 captured by the image capturer 91 at the second time t₁ includes a second left pupil ellipse 1808. The second right and left pupil ellipses 1806, 1807 may be collectively referred to as a set pupil location set 1814.

In operation, the velocity comparer 937 of FIG. 9 overlays the first right eye image 1801 with the second right eye image 1805 to generate a right eye composite 1809. Further, the velocity comparer 937 overlays the first left eye image 1803 with the second left eye image 1807 to generate a left eye composite 1810. The velocity comparer 937 determines a first distance 1811 between the first right pupil ellipse 1802 and the second right pupil ellipse 1806. The velocity comparer 937 determines a second distance 1812 between the first left pupil ellipse 1804 and the second left pupil ellipse 1808. Further, the velocity comparer 937 determines a right eye velocity (e.g., the first eye velocity 81 of FIG. 8) by dividing the first distance 1811 over the elapsed time between the first time t₀ and the second time t₁. Additionally, the velocity comparer 937 determines a left eye velocity (e.g., the second eye velocity 82 of FIG. 8) by dividing the second distance 1812 over the elapsed time between the first time t₀ and the second time t₁. A user may then judge whether the left and right eye velocities are dissimilar enough to diagnose the patient with a concussion. In some examples, the velocity comparer 937 may compare the difference between the left and right eye velocities against normal eye velocity differences stored in the eye velocity difference log 94 to aid the user in making the diagnosis. In some such examples, the velocity comparer displays the comparison via the display 231.

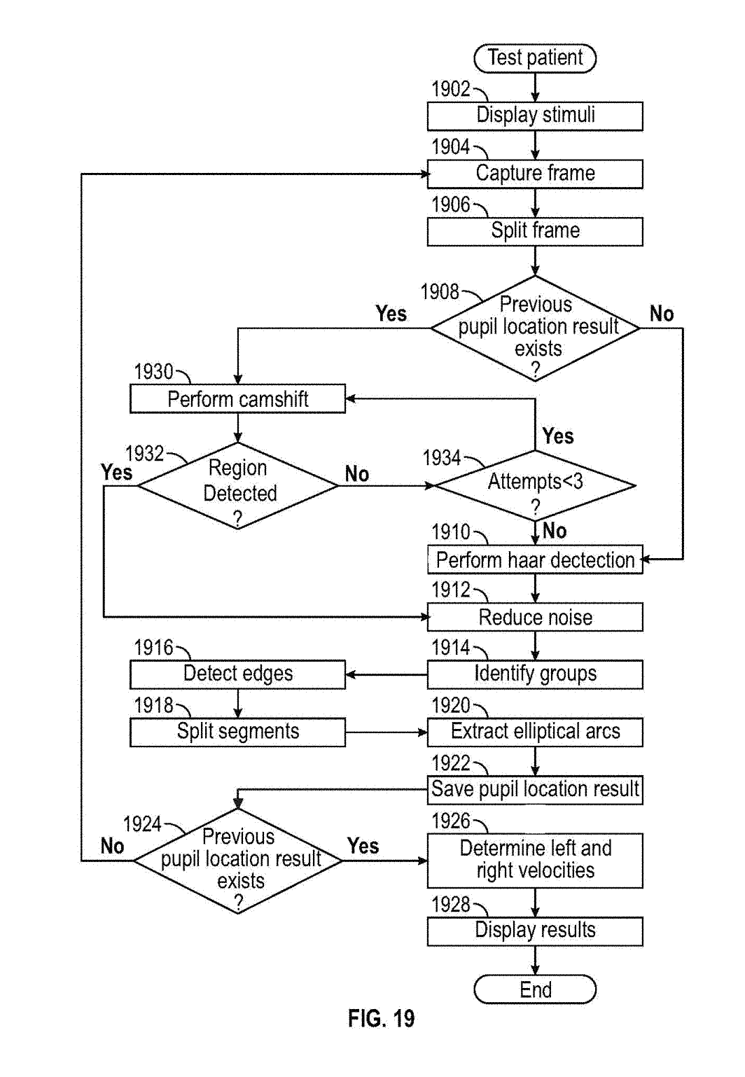

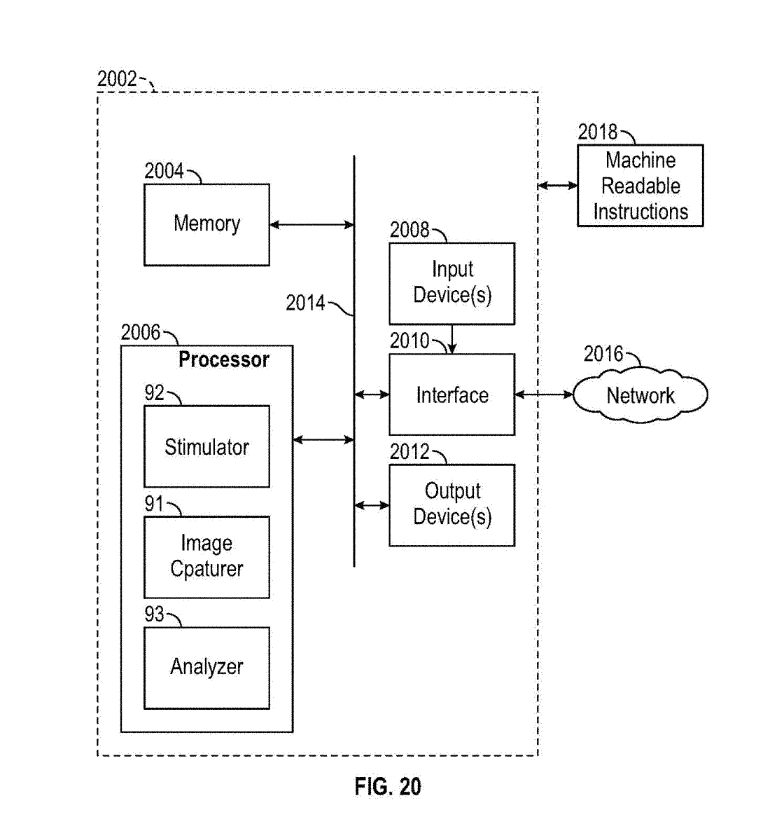

FIG. 19 is a flowchart of an example method 1900 to test a patient who may be concussed. The flowchart of FIG. 19 is representative of machine readable instructions that are stored in memory (such as the memory 2004 of FIG. 20) and include one or more programs which, when executed by a processor (such as the processor 2006 of FIG. 20), cause the mobile device 23 to implement the example ocular movement tester 9 of FIG. 9. While the example program is described with reference to the flowchart illustrated in FIG. 19, many other methods of implementing the example ocular movement tester 9 may alternatively be used. For example, the order of execution of the blocks may be rearranged, changed, eliminated, and/or combined to perform the method 1900. Further, because the method 1900 is disclosed in connection with the components of FIG. 9, some functions of those components will not be described in detail below.

The terms “non-transitory computer-readable medium” and “computer-readable medium” include a single medium or multiple media, such as a centralized or distributed database, and/or associated caches and servers that store one or more sets of instructions. Further, the terms “non-transitory computer-readable medium” and “computer-readable medium” include any tangible medium that is capable of storing, encoding or carrying a set of instructions for execution by a processor or that cause a system to perform any one or more of the methods or operations disclosed herein. As used herein, the term “computer readable medium” is expressly defined to include any type of computer readable storage device and/or storage disk and to exclude propagating signals.

In the illustrated example of FIG. 19, the example stimulator 92 of FIG. 9 displays the example visual stimulus 7 via the display 231 of the mobile device 23 of FIGS. 6-8 (block 1902). The example image capturer 91 of FIG. 9 captures a frame of the patient's eyes via the camera 232 of the mobile device 23 (block 1904). The example image capturer 91 then splits the frame into a set of left and right eye images (block 1906). The region detector 931 of FIG. 9 determines whether a set of previous pupil locations has already been found for a previous set of left and right eye images (block 1908).

If the region detector 931 determines that a set of previous pupil locations has already been found (block 1908), the region detector 931 performs a CAM shift on the left and right eye images using the set of previous pupil locations (block 1930). The region detector 931 determines whether a region including a pupil was detected via the CAM shift (block 1932).

If the region detector 931 determines that a region including a pupil was detected via the CAM shift (block 1932), the method 1900 proceeds to block 1912, to be explained greater detail below.

If the region detector 931 determines that a region including a pupil was not detected via the CAM shift (block 1932), the region detector 931 determines whether the CAM shift has been attempted a maximum three times (block 1934).

If the region detector 931 determines that the CAM shift has been attempted fewer than three times (block 1934), the method 1900 returns to block 1930.

If the region detector 931 determines that the CAM shift has already been attempted three times (block 1934), the method 1900 proceeds to block 1910.

Returning to block 1908, if region detector 931 determines that a set of previous pupil locations has not been found, the region detector 931 performs a Haar detection on each of the left and right images using the Haar-like feature 1201 to detect pupil locations in the left and right eye images (block 1910). The noise reducer 932 then reduces electronic noise in each of the left and right eye images (block 1912). The group identifier 933 then identifies groups 1110 in the left and right eye images based on pixel color in the left and right eye images (block 1914). The edge detector 934 then detects edges 1111 of the identified groups 1110 (block 1916). The segment splitter 935 then identifies corners 1112 of the edges 1111 and splits the detected edges 1111 into a plurality of segments 1113 at the corners 1112 (block 1918). The elliptical arc extractor 936 then generates quantized gradient direction distributions for each of the plurality of segments 1113 and filters out segments 1113 that have uneven quantized gradient direction distributions to extract a segment 1113 that is an elliptical arc 1114 for each of the left and right eye images (block 1920). The elliptical arc extractor 936 then saves where the elliptical arcs 1114 are located in each of the left and right eye images (block 1922). The velocity comparer 937 then determines whether a set of previous pupil locations for a previous set of left and right eye images has already been found (block 1924).

If the velocity comparer 937 determines that a set of previous pupil locations has not been found, the method 1900 returns to block 1904 to capture another frame.

If the velocity comparer 937 determines that a set of previous pupil locations has been found, the velocity comparer 937 determines left and right eye velocities based on distances between the left pupil locations and between the right pupil locations and the elapsed time between when the sets of left and right eye images were captured (e.g., the frame rate of the camera 232) (block 1926). The velocity comparer 937 then displays the left and right eye velocity results on the display 231. The method 1900 then ends.

FIG. 20 is a block diagram of an example computing platform 2002 capable of executing the instructions of FIG. 19 to implement the ocular movement tester 9 of FIG. 9. In the illustrated example of FIG. 20, the computing platform 2002 includes a memory 2004, a processor 2006, input device(s) 2008, an interface 2010, output device(s) 2012, and a bus 2014.

In the illustrated example of FIG. 20, the memory 2004, the processor 2006, the interface 2010, and the output device(s) 2012 are in communication with one another via the bus 2014. The input device(s) 2008 are in communication with the interface 2010.

In the illustrated example of FIG. 20, the processor 2006 of the on-board computing platform 2002 is structured to include the ocular movement tester 9. The processor 2006 may be any suitable processing device or set of processing devices such as, but not limited to, a microprocessor, a microcontroller-based platform, an integrated circuit, one or more field programmable gate arrays (FPGAs), and/or one or more application-specific integrated circuits (ASICs). The memory 2004 may be volatile memory (e.g., RAM including non-volatile RAM, magnetic RAM, ferroelectric RAM, etc.), non-volatile memory (e.g., disk memory, FLASH memory, EPROMs, EEPROMs, memristor-based non-volatile solid-state memory, etc.), unalterable memory (e.g., EPROMs), read-only memory, and/or high-capacity storage devices (e.g., hard drives, solid state drives, etc). In some examples, the memory 2004 includes multiple kinds of memory, particularly volatile memory and non-volatile memory.

The memory 2004 is computer readable media on which one or more sets of instructions 2018, such as the software for operating the methods of the present disclosure, can be embedded. The instructions 2018 may embody one or more of the methods or logic as described herein. For example, the instructions 2018 reside completely, or at least partially, within any one or more of the memory 2004, the computer readable medium, and/or within the processor 2006 during execution of the instructions 2018.

The interface 2010 may be implemented by any type of interface standard (e.g., Ethernet, universal serial bus (USB), and/or a peripheral component interconnect (PCI) express interface). The interface 2010 includes a communication device (e.g., a transmitter, a receiver, a transceiver, a modem, network interface card, etc.) to exchange data with external machines and/or computing devices via a network 2016 (e.g., an Ethernet connection, wireless connection, a telephone line, coaxial cable, a cellular telephone system, etc.).

The machine readable instructions 2018 of FIG. 19 may be stored in the memory 2004 and/or on a removable tangible computer readable medium storage (e.g., a compact disc, a digital versatile disc, a Blu-ray disc, a USB drive, etc.).

In the illustrated example of FIG. 20, the input device(s) 2008 enable a user, such as an operator or technician, to provide instructions, commands, and/or data to the processor 2006. Examples of the input device(s) 2008 include one or more of a button, a control knob, an instrument panel, a touch screen, a touchpad, a keyboard, a mouse, a speech recognition system, etc.

The output device(s) 2012 of the illustrated example display output information and/or data of the processor 2006 to a user, such as an operator or technician. Examples of the output device(s) 2012 include a liquid crystal display (LCD), an organic light emitting diode (OLED) display, a flat panel display, a touch screen, a solid state display, and/or any other device that visually presents information to a user. Additionally or alternatively, the output device(s) may include one or more speakers and/or any other device(s) that provide audio signals for a user. Further, the output device(s) 2012 may provide other types of output information, such as haptic signals.

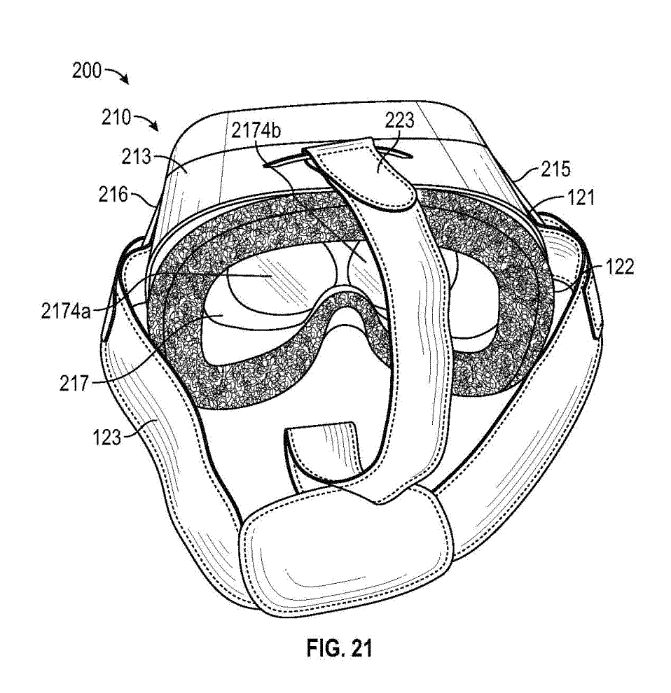

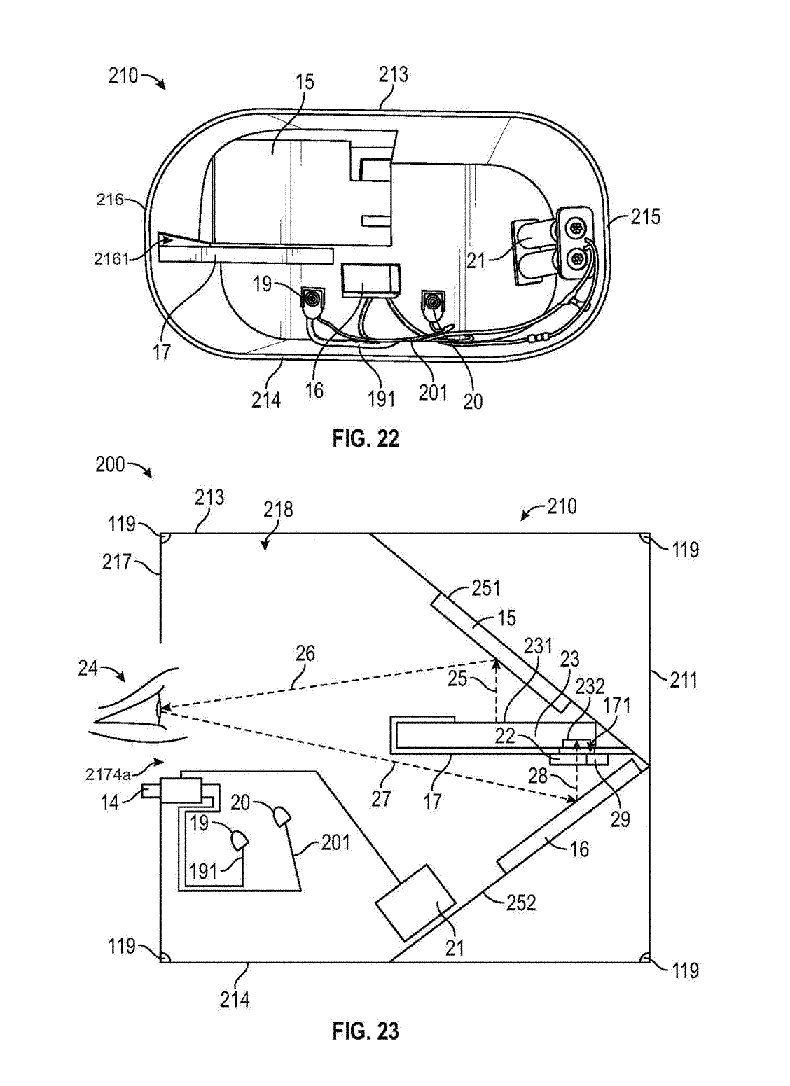

FIGS. 21, 22, and 23 illustrate a second example embodiment of a concussion testing device 200 as disclosed herein. In the illustrated examples of FIGS. 21, 22, and 23, the concussion testing device 200 includes a housing 210, the cushion 122, the strap 123, a stabilizing strap 223, the switch 14, the first mirror 15, and the second mirror 16. The housing 210 includes a first supporting rib 251, a second supporting rib 252, a front wall 211, a top wall 213, a bottom wall 214, a first side wall 215, a second side wall 216, and a patient-facing wall 217. In the illustrated examples of FIGS. 21 and 22, the first and second side walls 215, 216 are curved. The bottom wall 214 is curved to accommodate a patient's nose. The patient-facing wall 217 defines a first viewing port 2174a and a second viewing port 2174b. The second side wall 216 defines a mobile device port 2161.

As shown in the illustrated examples of FIGS. 21, 22, and 23, the top wall 213 is engaged with the first supporting rib 251, with the first side wall 215, with the second side wall 216, with the front wall 211, and with the patient-facing wall 217. The first supporting rib 251 is engaged with the top wall 213 and the front wall 211. The second supporting rib 252 is engaged with the bottom wall 214 and the front wall 211. The patient-facing wall 217 is engaged with the top wall 213, with the bottom wall 214, with the first side wall 215, and with the second side wall 216. In the illustrated examples of FIGS. 21, 22, and 23, the housing 210 is formed of molded plastic. It should be appreciated that the housing 210 may be formed of any sheet-type material (e.g., cardboard, sheet metal, plywood, foam board, etc.). The walls 211, 213, 214, 215, 216, 217 and the supporting ribs 251, 252 of the housing 210 may be engaged with one another with glue and/or with mechanical fasteners (e.g., staples, brads, nails, screws, pins, etc.). The walls 211, 213, 214, 215, 216, 217 and the supporting ribs 251, 252 of the housing 210 may be integrally formed with one another from sheet material and folded to form the housing 210.

As shown in the illustrated examples of FIGS. 21, 22, and 23, the cushion 122 is connected to the patient-facing wall 217. The strap 123 is engaged to the first and second sides 215, 216. The stabilizing strap 223 is engaged to the top wall 213 and the strap 123. The stabilizing strap 223 may be wrapped across the top of a patient's head to hold the concussion testing device 200 firmly against the patient's face. In the examples of FIGS. 21 and 22, the stabilizing strap 223 is an elasticized band. It should be appreciated that the stabilizing strap 223 may be formed of any lashing-type material (e.g., string, ribbon, leather, webbing, etc.).

In the illustrated example of FIG. 23, the switch 14 is carried by the patient facing wall 217. In this second example embodiment, the switch 14 is a contact (e.g., plunger style) switch that turns on the first and second focused red LEDs 19, 20 when a patient's face is pressed against the concussion testing device 200.

In the illustrated examples of FIGS. 22 and 23, the concussion testing device 200 further includes the mobile device bracket 17, the first focused red LED 19, the second focused red LED 20, the battery 21, the red light filter 22, and a blue light filter 29. The housing 210 defines a cavity 218.

The first mirror 15 is engaged with and supported by the first supporting rib 251. The second mirror 16 is engaged with and supported by the second supporting rib 252. The mobile device bracket 17 is engaged with and supported by the second wall 216 and the front wall 211. The mobile device bracket 17 is substantially perpendicular to the patient-facing wall 217. Seams formed between the walls 211, 213, 214, 215, 216, 217 of the housing 210 may be sealed with the light-blocking sealant 119.

As above, the mobile device bracket 17 removably engages and supports a mobile device 23. The mobile device 23 is inserted into the mobile device bracket 17 via the mobile device port 2161. The mobile device bracket 17 is engaged with and supports the red light filter 22 and the blue light filter 29. In this second example embodiment, the red light filter 22 and the blue light filter 29 cover the camera opening 171. In some examples, the blue light filter 29 is formed of a translucent gel. Further, the mobile device 23 is removed from the mobile device bracket 17 via the mobile device port 2161.

In this second example embodiment, the battery 21 is in electrical communication with the switch 14. The switch 14 is in electrical communication with the first focused red LED 19 via the first supporting wire 191 and with the second focused red LED 20 via the second supporting wire 201. The first supporting wire 191 holds the first focused red LED 19 up in the cavity 218 near the first patient eye 24. The second supporting wire 201 holds the second focused red LED 20 up in the cavity 218 near a second patient eye (not shown).

In operation, the first and second focused red LEDs 19, 20 are energized by the battery 21 to provide a red light source within the housing 210. In operation, a LED of the mobile device 32 is illuminated to shine through the blue light filter 29 to provide a blue light source within the housing 210. Thus, blue light is diffused throughout the cavity 218. The first focused red LED 19 directs red light to the first patient eye 24. The second focused red LED 20 directs red light to the second patient eye. Further, in operation, the visual stimulus 7 is displayed on the display 231, as explained above. The visual stimulus 7 bounces off the first mirror 15 to be observed by the first patient eye 24 and by the second patient eye via the first and second viewing ports 2174a, 2174b, as indicated by dashed lines 25, 26 and explained above. The camera 232 records responses of the first patient eye 24 and of the second patient eye to the visual stimulus 7 via the second mirror 16, as indicated by dashed lines 27, 28 and explained above.

From the foregoing, it will be appreciated that the above disclosed methods and apparatus may aid in diagnosing players for concussions substantially accurately and promptly. Thus, concussed athletes may be removed from play for treatment while healthy (e.g., not concussed) athletes may return to play. Further, the above disclosed methods and apparatus significantly reduce the cost and size of concussion diagnosis devices, thus encouraging widespread evaluation of potential concussions. Additionally, the above disclosed methods and apparatus provide a specific improvement to computer-related technology by reducing the amount of image processing needed to determine specific shapes (e.g., ellipses) from low-contrast edges (e.g., between a pupil and an iris), thus freeing a processor to perform other tasks more quickly and consuming less energy.

Although certain example methods, apparatus, and articles of manufacture have been disclosed herein, the scope of coverage of this patent is not limited thereto. On the contrary, this patent covers all methods, apparatus, and articles of manufacture fairly falling within the scope of the claims of this patent.

What is claimed is:

1. An apparatus comprising:
a housing having a viewing port;
a mobile device bracket disposed in the housing and configured to support a mobile device, the mobile device having a display and a camera;
a first mirror disposed in the housing to reflect the display toward the viewing port;
a second mirror disposed in the housing to reflect the viewing port toward the camera; and
a light source disposed in the housing.

2. The apparatus of claim 1, wherein the light source comprises a focused red light emitting diode.

3. The apparatus of claim 1, further comprising a red light filter to filter light received by the camera.

4. The apparatus of claim 1, wherein the light source comprises a diffused blue light emitting diode.

5. The apparatus of claim 4, further comprising a potentiometer to regulate an intensity of the diffused blue light emitting diode.

6. The apparatus of claim 1, wherein the housing comprises a side wall, the side wall having a mobile device port to insert the mobile device into the mobile device bracket.

7. The apparatus of claim 1, wherein the housing comprises a first sloped wall and a second sloped wall, the first sloped wall supporting the first mirror, and the second sloped wall supporting the second mirror.

8. An apparatus comprising:
a stimulator to provide a visual stimulus to a patient eye;
an image capturer to capture visual data of the patient eye in response to the visual stimulus;
an elliptical arc extractor to determine a first set of pupil locations corresponding to a first time and a second set of pupil locations corresponding to a second time based on the visual data; and
a velocity comparer to determine a first pupil velocity and a second pupil velocity based on the first and second pupil location sets and the first and second times.

9. The apparatus of claim 8, further comprising a region detector to detect a region in the visual data containing a pupil.

10. The apparatus of claim 8, further comprising a group identifier to identify a plurality groups based on pixel colors in the visual data.

11. The apparatus of claim 10, further comprising an edge detector to detect edges of the plurality of groups.

12. The apparatus of claim 11, further comprising a segment splitter to detect corners of the edges and to split the edges into a plurality of segments at the corners.

13. The apparatus of claim 12, wherein:
the elliptical arc extractor generates a plurality of quantized gradient direction distributions corresponding to the plurality of segments based on gradient vectors of pixels along the plurality of segments; and
the first and second pupil location sets correspond to ones of the plurality of quantized gradient direction distributions that are relatively even.

14. The apparatus of claim 13, wherein the gradient vectors are based on color differences between neighboring pixels of the pixels along the plurality of segments.

15. A method comprising:
providing, by executing an instruction with a processor, a visual stimulus to a patient eye;
capturing, by executing an instruction with the processor, visual data of the patient eye in response to the visual stimulus;
determining, by executing an instruction with the processor, a first set of pupil locations corresponding to a first time and a second set of pupil locations corresponding to a second time based on the visual data; and
determining, by executing an instruction with the processor, a first pupil velocity and a second pupil velocity based on the first and second pupil location sets and the first and second times.

16. The method of claim 15, further comprising detecting, by executing an instruction with the processor, a region in the visual data containing a pupil.

17. The method of claim 15, further comprising identifying, by executing an instruction with the processor, a plurality groups based on pixel colors in the visual data.

18. The method of claim 17, further comprising detecting, by executing an instruction with the processor, edges of the plurality of groups.

19. The method of claim 18, further comprising:
detecting, by executing an instruction with the processor, corners of the edges; and
splitting, by executing an instruction with the processor, the edges into a plurality of segments at the corners.

20. The method of claim 19, wherein:
determining, by executing an instruction with the processor, the first set of pupil locations and the second set of pupil locations comprises generating a plurality of quantized gradient direction distributions corresponding to the plurality of segments based on gradient vectors of pixels along the plurality of segments; and
the first and second pupil location sets correspond to ones of the plurality of quantized gradient direction distributions that are relatively even.

21. The method of claim 20, wherein the gradient vectors are based on color differences between neighboring pixels of the pixels along the plurality of segments.
